(19) United States
(12) Patent Application Publication
(10) Pub. No.: US 2025/0166770 A1
(43) Pub. Date: May 22, 2025

(54) SYSTEM AND METHOD FOR GENERATING A BODY MODIFICATION PROTOCOL

(71) Applicants: Ronen Yair, Millburn, NJ (US); Eva Allan, New York, NY (US)

(72) Inventors: Ronen Yair, Millburn, NJ (US); Eva Allan, New York, NY (US)

(21) Appl. No.: 18/948,622

(22) Filed: Nov. 15, 2024

Related U.S. Application Data
(60) Provisional application No. 63/601,977, filed on Nov. 22, 2023.

(51) Int. Cl. G16H 20/00; G06T 17/00; G06V 10/82; G06V 40/10; G16H 15/00; G16H 50/20

(52) U.S. Cl. CPC G16H 20/00; G06V 10/82; G06V 40/10; G16H 50/20

(57) ABSTRACT

A system for generating personalized body modification protocols includes utilizing one or more image capture devices, such as smartphones or camera arrays. The system captures the user's current physical attributes. Integrated with the image capture device is a machine learning model, including a neural network and a computer with a processor and memory. The computer assesses the user's physical state. Additional images of another individual can be processed as a desired physique benchmark for the user. The neural network contrasts the user's existing and aspired bodily states. Based on this comparison, the system identifies potential body modification options, and provides the user with a customized protocol to align closer to the user's desired physical appearance.

### CROSS-REFERENCE TO RELATED APPLICATION

The present application claims priority to U.S. Provisional Patent Application No. 63/601,977, filed on Nov. 22, 2023, the entire contents of which are incorporated by reference herein.

### FIELD

The present disclosure relates to a system and method of providing a multi-faceted use-specific protocol, and more particularly, to a system and method of delivering a customized body modification protocol to a user.

### BACKGROUND

In contemporary society, a number of body modification methods are extensively adopted by individuals in pursuit of their perceived idealistic aesthetic. Such modifications encompass a spectrum of approaches, ranging from dietary plans, surgical interventions, physical exercise routines, dental enhancements, the application of cosmetic products, to skin care routines. It's not uncommon for individuals to amalgamate several of these modalities, among others, in their pursuit of achieving a particular appearance.

Furthermore, societal influences, especially from popular culture and media, often amplify the desire for these aesthetic transformations. Utilizing these protocols, either in isolation or in combination, enables users to improve their bodily appearance, physical health, and/or mental health. As such, there is a burgeoning demand for holistic systems to consolidate and streamline the various facets of body modification to offer an individualized body modification protocol.

### SUMMARY

The system and method described herein are designed to generate a tailored body modification protocol for users. A singular or multiple image capture devices, for example, a smartphone or camera array, may be employed to obtain a visual representation of the user's current physical bodily state. A machine learning model is in communication with the image device or devices. The machine learning module includes a neural network and a computer. Upon receiving one or more images of a user's body from the image capture device or devices, the computer is configured to assess the user's existing physical attributes. The computer may receive and process images of another individual, serving as a benchmark for the user's modification aspirations. The neural network then engages in a comparative analysis between the user's present bodily state and the user's desired body alterations. Following a comparative analysis, the neural network may identify a range of feasible potential body modification procedures designed to guide a user closer to their envisioned physique or appearance. The system offers a comprehensive customized body modification plan for a user to follow to draw the user closer to their desired physical appearance.

Provided in accordance with aspects of the present disclosure is a system for generating a body modification protocol including one or more image capture devices. The image capture device is configured to capture at least one image of a user's body. A machine learning model is in communication with the image capture device. The machine learning model includes a neural network. The system includes a computer, a processor and a memory. The computer is in communication with the image capture device(s) and the machine learning model. The memory stores computer instructions configured to instruct the processor to receive at least one image of the user's body from the image capture device(s). The memory stores computer instructions configured to instruct the processor to determine a current body configuration for the user based on the image(s) received from the image capture device. The memory stores computer instructions configured to instruct the processor to receive at least one image of a body of another person from the user. The memory stores computer instructions configured to instruct the processor to determine a desired body configuration for the user based on the image(s) of the body of another person. The memory stores computer instructions configured to instruct the processor to compare, by the neural network, the current body configuration for the user with the desired body configuration for the user. The memory stores computer instructions configured to instruct the processor to determine, by the neural network, a number of available body modification procedures that could be employed by the user to attain the desired body configuration with respect to the current body configuration for the user. The memory stores computer instructions configured to instruct the processor to deliver a body modification protocol to the user. The body modification protocol delivered to the user includes at least some of the available body modification procedures for attaining the desired body configuration for the user.

In an aspect of the present disclosure, the body modification procedures include an exercise regimen, a diet regimen, a meal plan, a surgical intervention, a non-surgical intervention, a medical intervention, an injection, a facial, a laser treatment, a dental procedure, a salon procedure, a self-care procedure, and/or a cosmetic application procedure.

In an aspect of the present disclosure, the computer is configured to instruct the processor to receive at least one user-preferred body modification procedure. Each of the available body modifications procedures is assigned a first weight. At least one of the user-preferred body modification procedures receives a higher valued second weight. The neural network then determines the available body modification procedures based on the first weight applied to the available body modification procedures and the second weight applied to user-preferred body modification procedure(s).

In an aspect of the present disclosure, the computer is configured to instruct the processor to receive at least one user-disfavored body modification procedure. The user-disfavored body modification procedure(s) are assigned a third weight. The third weight is lower than the first weight. The neural network determines the available modification procedures based on the first weight applied to the body modification procedures and the third weight applied to disfavored body modification procedure(s).

In an aspect of the present disclosure, the computer instructions are configured to instruct the processor to identify at least one body modification procedure that would be unsafe. The computer instructions are configured to instruct the processor to exclude the unsafe body modification procedures.

In an aspect of the present disclosure, the computer instructions are configured to instruct the processor to receive characteristic data for the user. The characteristic data includes demographic information, medical history, family medical history, comorbidities, allergies or allergy, medication use, pregnancy status, number of children, activity level, and/or body dimensions. The neural network determines the available body modification procedures based on the received user's characteristic data.

In an aspect of the present disclosure, the computer instructions are configured to instruct the processor to receive user ratings for at least one particular portion of the user's body. User rating may be for one or more of the user's: hair, face, skin, facial structure, smile, chin, neck, arm, underarm, chest, breasts, back, tummy, hip, buttocks, genitals, flank, thigh, knee, calf, ankle, or feet. The neural network determines the available body modification procedures based on the received user ratings for the portion(s) of the user's body.

In an aspect of the present disclosure, the image capture device is configured to capture a number of images of the user's body. The computer instructions are configured to instruct the processor to stitch together the images of the user's body to create a 3-dimensional model of the user's body.

In an aspect of the present disclosure, the machine learning module includes a convolutional neural network (CNN) configured to analyze one or more of the images of the user's body to determine the user's current body configuration and one or more image of the body of another person to determine the desired body configuration for the user.

In an aspect of the present disclosure, the machine learning module includes a chatbot module that may be configured to receive user feedback and provide responses to the user. The responses are configured to assist the user in completing the body modification protocol and further tailor the body modification protocol to meet the user's aspirations. Furthermore, the chatbot may be configured to receive user input, such as areas of concern, preferences, and disfavored body modification procedures.

In an aspect of the present disclosure, a computer-implemented method of generating a body modification protocol includes receiving one or more image of the user's body from at least one image capture device. The image or images received from the user are used to determine the user's current body configuration. At least one image of a body of another person is received from the user. The user's desired body configuration is determined based on the images(s) of the body of another person chosen by the user. The user's current body configuration is compared with the desired body configuration for the user by the neural network. The available body modification procedures that could be employed by the user to attain the desired body configuration with respect to the current body configuration for the user are determined by the neural network. A body modification protocol is delivered to the user. The body modification protocol delivered to the user includes at least some of the available body modification procedures for attaining the desired body configuration for the user.

In an aspect of the present disclosure, the computer implemented method includes receiving at least one preferred body modification procedure from the user. The method includes applying a first weight to each of the available body modification procedures. The method includes applying a second weight to the preferred body modification procedure(s). The second weight is higher than the first weight. The available body modification procedures are determined, by the neural network, based on the first weight applied to the available body modification procedures and the second weight applied to the preferred body modification procedure(s).

In an aspect of the present disclosure, the computer implemented method includes receiving at least one disfavored body modification procedure from the user. A third weight is applied to the disfavored body modification procedure(s). The third weight is lower than the first weight. The available body modification procedures are determined, by the neural network, based on the first weight applied to the available body modification procedures and the third weight applied to the disfavored body modification procedure(s).

In an aspect of the present disclosure, the computer implemented method includes identifying at least one available body modification procedure that would be unsafe for the user. The method includes excluding the identified body modification procedure that would be unsafe for the user from the body modification protocol delivered to the user.

In an aspect of the present disclosure, the computer implemented method includes receiving characteristic data for the user. The received characteristic data for the user includes demographic information, a medical history for the user, a family medical history for the user, a comorbidity for the user, an allergy for the user, a medication consumed by the user, a pregnancy status for the user, a number of children had by the user, an activity level for the user, and/or dimensions of the user's body measured by the user. The available body modification procedures are determined by the neural network based on the received characteristic data for the user.

In an aspect of the present disclosure, the computer implemented method includes receiving user ratings for at least one particular portion of the user's body. The particular portion(s) of the user's body includes the user's hair, face, skin, facial structure, smile, chin, neck, arm, underarm, chest, back, tummy, hip, flank, thigh, knee, calf, ankle, and/or feet. The method includes determining the available body modification procedures, by the neural network, based on the received user ratings for the portion(s) of the user's body.

In an aspect of the present disclosure, the computer implemented method includes capturing, by the image capture device, images of the user's body, and stitching together the images of the user's body to create a 3-dimensional model of the user's body.

### BRIEF DESCRIPTION OF THE DRAWINGS

Various aspects and features of the present disclosure are described hereinbelow with reference to the drawings wherein:

FIG. 1A is a schematic diagram of a system for generating a customized body modification protocol according to aspects of the present disclosure;

FIG. 1B is a schematic diagram of another system for generating a customized body modification protocol according to aspects of the present disclosure;

FIG. 2 is a schematic diagram of an exemplary neural network architecture employable by the system of FIGS. 1A or 1B;

FIG. 3 is a flowchart of a computer-implemented method of providing a customized body modification protocol to a user according to aspects of the present disclosure;

FIG. 4 is a flowchart of another computer-implemented method of providing a customized body modification protocol according to aspects of the present disclosure;

FIG. 5 is a flowchart of another computer-implemented method of providing a customized body modification protocol according to aspects of the present disclosure;

FIG. 6 is a flowchart of another computer-implemented method of providing a customized body modification protocol according to aspects of the present disclosure;

FIG. 7 is a flowchart of another computer-implemented method of providing a customized body modification protocol according to aspects of the present disclosure;

FIG. 8 is a flowchart of another computer-implemented method of providing a customized body modification protocol according to aspects of the present disclosure;

FIG. 9 is a flowchart of another computer-implemented method of providing a customized body modification protocol according to aspects of the present disclosure;

FIG. 10 is a block diagram of an exemplary computer for implementing the method of providing a customized body modification protocol according to aspects of the present disclosure;

FIG. 11 is a flowchart of another computer-implemented method of providing a customized body modification protocol to a user according to aspects of the present disclosure; and

FIG. 12 is a flowchart of another computer-implemented method of providing a customized body modification protocol to a user according to aspects of the present disclosure.

### DETAILED DESCRIPTION

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings). Like reference numerals may refer to like elements throughout the specification and drawings.

The customized body modification protocol system (CBMPS) described herein (e.g., system 100) is designed to integrate a comprehensive range of data about a user. This includes details pertaining to their health, current body image, and images representing their desired aesthetic goals. In response to this input, the CBMPS can execute two exemplary functions. First, it generates a simulation that provides a visual representation of the user's potential appearance modification, taking into consideration the efficacy of contemporary treatments and how closely the simulation aligns with the user's ideal aesthetic. Second, the CBMPS formulates a cross-disciplinary treatment plan, seamlessly integrating treatments from various disciplines. This plan is meticulously curated, reflecting the user's stated preferences, feasibility, and the user's expressed willingness to undergo specific procedures.

The CBMPS can employ a multitude of disciplines to assist the user in attaining their desired physical appearance. These disciplines can include, but are not limited to, exercise, diet, meal planning, surgical interventions (including setting appointments, estimating recovery times, recommending and/or procuring recovery supplies, rehabilitation, and/or massage), non-surgical intervention (e.g., CoolSculpting, Emsculpt, laser lipolysis, ultrasound fat reduction, microneedling, chemical peels, HydraFacial, microdermabrasion, thread lift, varicose and spider vein treatments, lymphatic drainage massage, infrared saunas and photofacials), medications (e.g. Adipex®, Suprenza®), injections (e.g., Botox®, dermal fillers, and Kybella®), facials and lasers, dental work, salon services (e.g., hair and nail services, hair removal, eyebrow services, and tanning), self-care (e.g., creams, cleansers, exfoliators, masks, toners, serums and oils, hair masks, saunas and steam rooms), and make-up. The CBMPS can enhance the user's bodily appearance, physical health, or mental health or a combination thereof.

In an exemplary embodiment of the disclosed system, user preferences are thoroughly considered to optimize the outcomes tailored to the individual's needs and desires. The system assesses the user's inclination towards the speed of obtaining results, factoring in how quickly they wish to see changes. Concurrently, it evaluates the user's budgetary constraints, determining user comfort with cost thresholds. The system also considers the user's openness to modifying their dietary habits, gauging their willingness to embrace dietary changes as a pathway to achieving their goals. Another facet considered is the user's proclivity towards engaging in physical activities, discerning the types and intensity of exercise the user is inclined to pursue. Surgical interventions are also weighed, assessing the user's readiness for such procedures. In parallel, non-surgical technological treatments are evaluated, as well as the potential utilization of injectables, such as Botox® and dermal fillers. The system further contemplates the user's openness to medications and the potential incorporation of hormones like testosterone. The system examines the degree of similarity the user seeks in comparison to their desired appearance, determining whether they aim for a whole or partial resemblance as applicable. Through this comprehensive assessment, the system ensures a personalized approach in recommending interventions that align closely with the user's preferences.

The CBMPS is adeptly designed to safeguard the user's well-being by integrating crucial constraints tailored to the user's health and safety. Foremost, the system has the capability to restrict treatments that might potentially exacerbate pre-existing health conditions of the user, ensuring that their overall health is not detrimentally impacted. As required, physicians or other healthcare of licensed providers supervise, prescribe or perform the procedures described herein. The system will facilitate scheduling of these consultations. Additionally, the CBMPS limits certain extreme surgical procedures, such as bone reconstruction. The system's inherent safety mechanisms further extend to precluding activities and treatments deemed to be hazardous to the user's health. This may include, but is not limited to, practices like extreme tanning, extreme starvation diets, illicit drug use, procedures that have recently come under scrutiny due to concerns about their safety, and the use of drugs not sanctioned by the FDA. When confronted with user preferences regarding cost and speed that are assessed as implausible or hazardous, the system does not simply reject these preferences. Instead, it proactively communicates the associated risks to the user and proposes an alternative plan that is more in alignment with the user's safety and overall well-being, ensuring informed decision-making and optimal outcomes. The system prevents the overuse of particular procedures based on the most current clinical guidelines and known best medical practices (e.g., too many Botox® injections in give period of time).

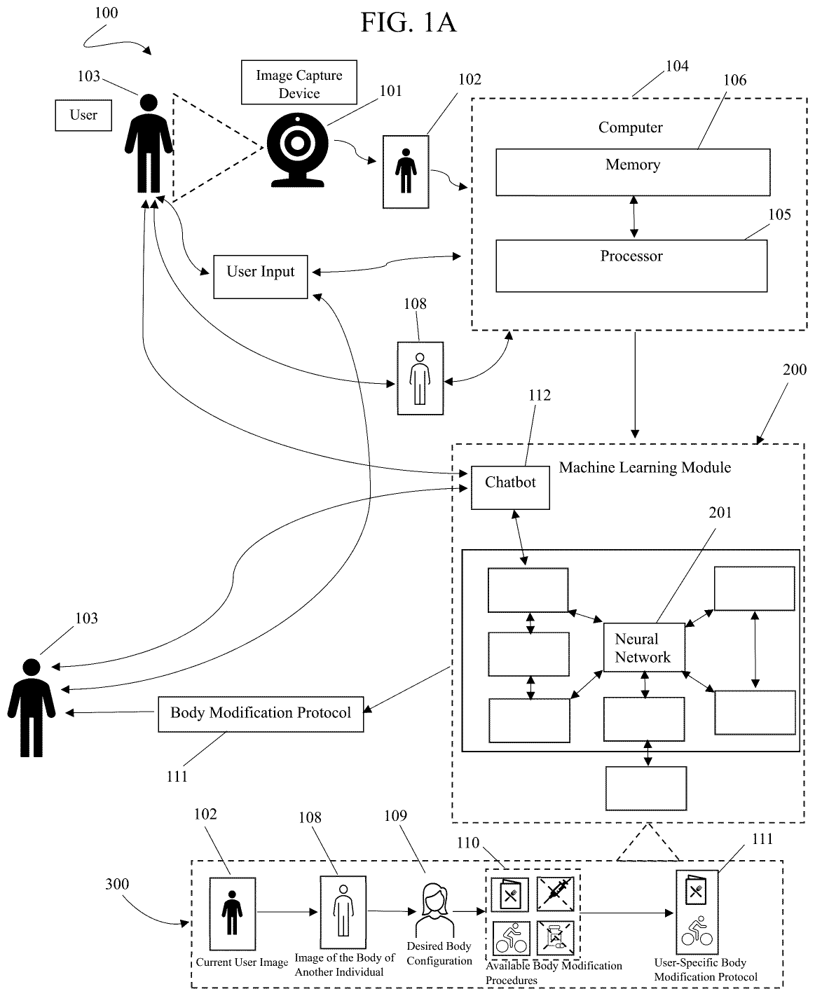

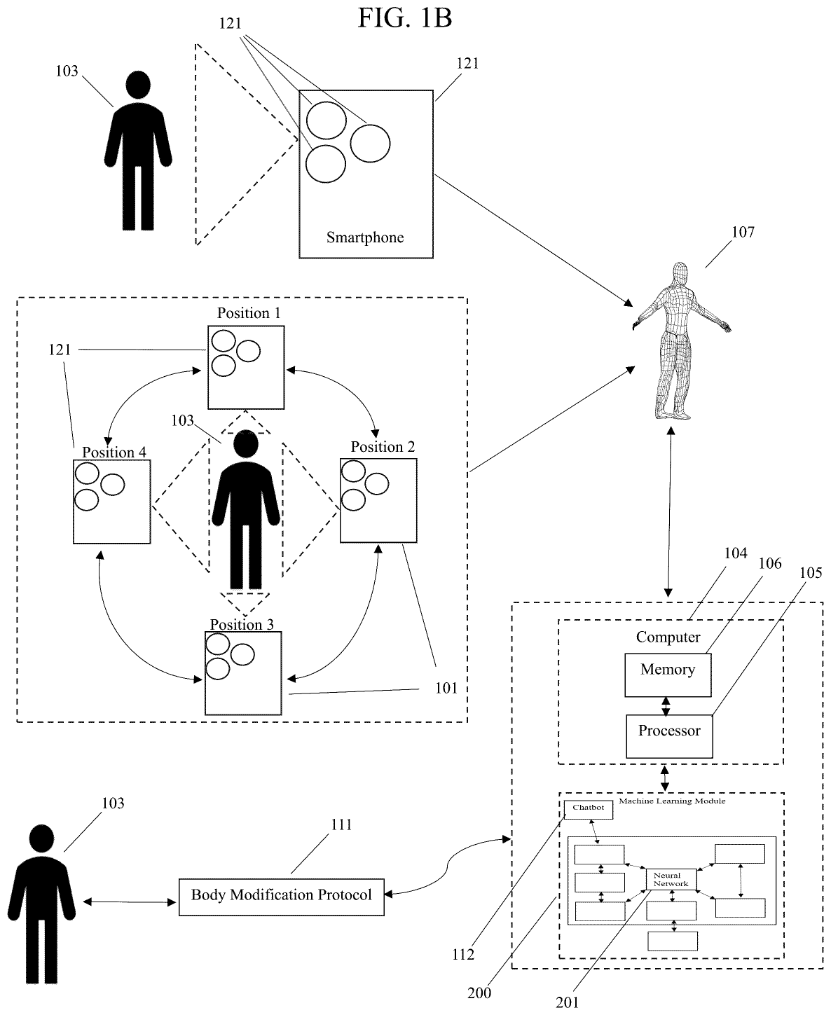

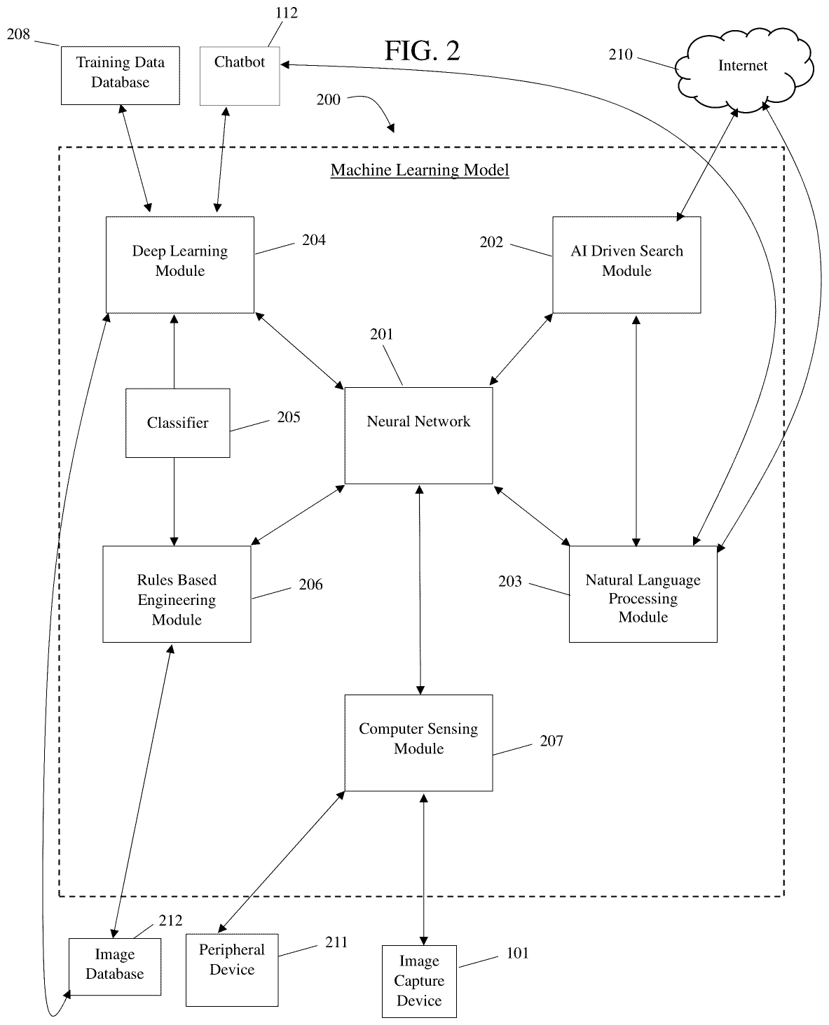

Referring particularly to FIGS. 1A, 1B, and 2, a system 100 for generating a body modification protocol includes one or more image capture devices 101. The image capture device 101 is configured to capture at least one image 102 of a user's body 103.

The image capture device described herein (e.g., image capture device 101 or image capture device 121) may be a standalone hardware device, or an image capture device included in a smartphone, tablet computer or laptop computer. For example, the image capture device may be a single digital camera, a digital camera array (see, e.g., camera array 122 in FIG. 1B), or a digital video camera.

A machine learning model 200 is in communication with the image capture device 101. The machine learning model 200 includes a neural network 201. The machine learning model 200 is described in more detail below with particular reference to FIG. 1

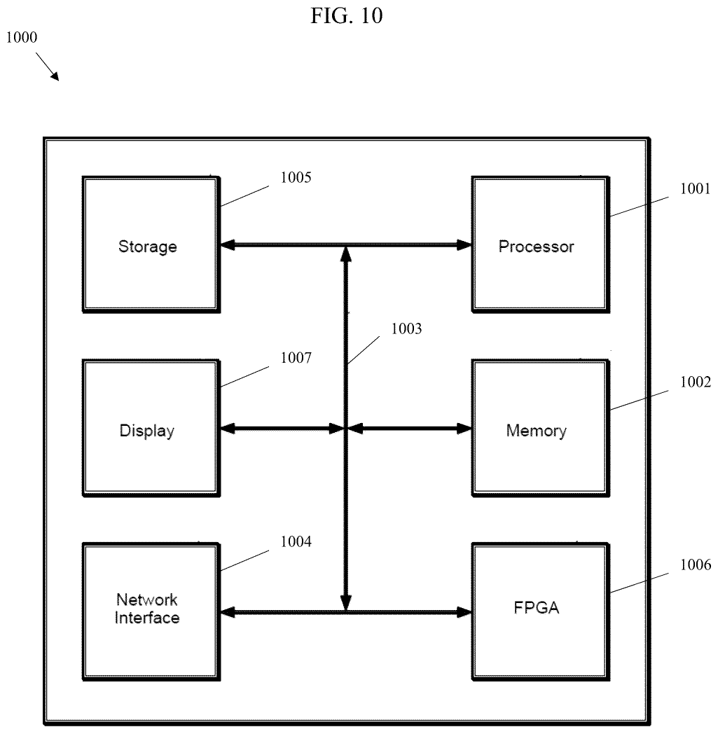

The system 100 includes a computer 104, a processor 105, and a memory 106. An exemplary computer structure (see, e.g., computer 1000) is described in more detail below with particular reference to FIG. 10. The computer 104 is in communication with the image capture device(s) 101 and the machine learning model 200.

The memory 106 stores computer instructions configured to instruct the processor 105 to receive at least one image 102 of the user's body 103 from the image capture device(s) 101. The memory 106 stores computer instructions configured to instruct the processor 105 to determine a current body configuration for the user (see, e.g., user model 107 in FIG. 1B) based on the image(s) 102 received from the image capture device 101. The memory 106 stores computer instructions configured to instruct the processor 105 to receive at least one image 107 of a body of another person from the user. The memory 106 stores computer instructions configured to instruct the processor 105 to determine a desired body configuration for the user 109 based on the image(s) of the body of another person. As an example, the desired body configuration for the user 109 may be a 3-D model similar to the model 107.

The memory 106 stores computer instructions configured to instruct the processor 105 to compare, by the neural network 201, the current body configuration for the user with the desired body configuration for the user. The memory 106 stores computer instructions configured to instruct the processor 105 to determine, by the neural network 201, a number of available body modification procedures 110 that could be employed by the user 103 to attain the desired body configuration 109 with respect to the current body configuration 107 for the user 103. The memory 106 stores computer instructions configured to instruct the processor 105 to deliver a body modification protocol 111 to the user 103. The body modification protocol 111 delivered to the user 103 includes at least some of the available body modification procedures 110 for attaining the desired body configuration for the user 103. The body modification protocol 111 is generated by the neural network 201, and the generated body modification protocol 111 is customized or personalized for the particular user, as described herein.

The body modification protocol 111 may include a schedule that accounts for a sequence of procedures, their recovery times, their pre and post-care steps, procedures which can be simultaneously overlaid, average costs and financing. The schedule may be a calendar, such as a digital calendar of a device employed by the user. The schedule or calendar may include reminders to book/schedule or attend the procedures of the body modification protocol with a fitness or healthcare provider, and/or to follow up with a fitness or healthcare provider.

The body modification protocol 111 may be customized to account for time constraints communicated by the user. For example, a user may only want a body modification protocol that can achieve the desired results within a limited period of time (e.g., within 1-week, 1-month, 6-months, etc.).

An exemplary architecture of the machine learning model employable by the system 100 is illustrated in FIG. 2. As an example, and referring particularly to FIG. 2, the machine learning model 200 may include the neural network 201 (i.e., an artificial neural network) including or configured to communicate with a deep learning module 204, a classifier 205, a rules based engineering module 206, a computer sensing module 207, a natural language processing module 203, and/or an artificial intelligence (AI) drive search module 202. The Deep learning module 204 may access training data, such as training data stored in a training data database 208. The training data database 208 can be continuously updated with new or expanded training data. Training an AI module, such as a deep learning model, is described in more detail below. The classifier 205 may be employed by at least one of the deep learning module 204 or the rules based engineering module 206. The computer sensing module 207 may be employed in acquiring and analyzing intricate data pertaining to the user 103 (e.g., data about the user's body) and/or interfacing with the image capture device 101 to generate a holistic view of the use's 103 current physical state. The computer sensing module 207 may employ or interface with any of the image capture devices described herein (see, e.g., image capture device 101 in FIG. 1A or the smartphone, tablet or webcam 121 in FIG. 1B). The AI drive search module 202 and/or the natural language processing module 203 may communicate with the internet 210 to receive data employable in generating available body modification protocols. Updated information may be captured from the internet 210 on a constant and instantaneous or near-instantaneous basis, such that body modification protocols can always be maximally current with advancements in medicine and employed for use in generating body modification protocols (e.g., eliminating dangerous procedures or suggesting cutting edge procedures).

The neural network 201 may refer to the architectural core of the machine learning model 200. The neural network 201 may take a set of inputs, pass the inputs through a series of hidden layers, in which each layer can transform the inputs, and then produce an output. The process of transforming the input is determined by the weights and biases of the neurons in the hidden layers of the neural network 201, which are learned from data during training of the neural network (see, e.g., training data database 208). The neural network 201 may include relatively simple (single layer) or relatively complex structures (multiple layers). The deep learning module 204 may employ a particular type of neural network (e.g., a Convolutional Neural Network) to process image data, while the classifier 205 may use another type of neural network (e.g., a Feed-Forward Neural Network) to make predictions based on the processed data.

The deep learning module 204 may be employed by the neural network 201. The deep learning module 204 may deliver high-dimensional representations of user data to the neural network 201. The neural network 201 may then use the information from the deep learning module 204 to learn complex patterns and inform the neural network's decision-making processes. Similarly, the classifier 205 may be employed by the neural network 201. The classifier 205 may use the neural network's output to categorize or classify inputs into different classes. Additionally, the neural network 201 may help guide the AI-driven search module 202 by helping to understand and rank procedures according to the procedure's potential effectiveness in aiding a user. The AI-driven search module 202 may use the learned representations from the neural network 201 to better tailor suggested body modification protocols. The neural network 201 may work with the natural language processing module 203 by generating language representations that the natural language processing module 203 may use for understanding and generating text. The neural network may employ the imagery data from the computer sensing module 207 to help inform the neural network's understanding of the user's current bodily appearance. For example, imagery data from the computer sensing module 207 may be employed to adjust recommended protocol according to the user's current bodily appearance in comparison to the user's desired bodily appearance.

The computer sensing module 207 may process imagery data (e.g., digital image or digital video) received at the machine learning module 200. For example, if the user provides a 360-degree video of their physique, the computer sensing module 207 might stitch together these sequential images to create a comprehensive 3-dimensional model of the user's body, enabling more accurate assessments and recommendations in the modification protocol.

Real-time user feedback from the chatbot 112 can be used to further personalize the body modification protocol delivered to the user (e.g., a user may receive an altered exercise plan based on user complaints of soreness). User feedback may include treatments performed, updated results (weight loss/gain), which may be utilized to generate an updated set of results for the system to recalibrate/revise the body modification protocol. The chatbot 112 may be in communication with the natural language processing module 203 to understand natural spoken language from a user and to provide feedback or suggestions in the form of text or audio delivered to the user. This real-time user feedback can be used to affect the type of modification procedures chosen by the classifier 205. The real-time user feedback may be used by the rules-based engineering module 206 to modify the type of modification procedures suggested to the user. The rules-based engineering module 206 may execute one or more rule based algorithms relating to user data, for example, if the user is allergic to a particular diet plan. In that circumstance, the machine learning model 200 would adapt and exclude any recommendations that incorporate that specific diet, ensuring a personalized and safe body modification protocol tailored to the user's unique requirements.

Data from the computer sensing module 207 can be used by the AI-driven search module 202 to refine a body modification procedure. For example, if the computer sensing module 207 detects particular body features or attributes that a user is consistently focusing on, the AI-driven search module 202 might prioritize body modification procedures relevant to those specific features in its suggestions. Feedback, including verbal reactions or queries from the chatbot 112 regarding certain procedures, can be interpreted by the natural language processing module 203, and refine the system's recommendations to better align with the user's preferences and concerns.

The deep learning module 204 can be employed for generating embeddings and high-dimensional representations of the user's physical attributes and body modification preferences. The deep learning module 204 can receive data inputs such as user age, gender, body part ranking, genetic information and/or predispositions, interest level in diet or exercise plans, desired physical outcomes, and transform these inputs into a representation of the user's underlying preferences. The outputs from the deep learning module 204 can be employed by the other modules within the machine learning model 200 to tailor body modification protocols suited for the individual. Over the course of predictions and feedback, the deep learning module 204 can refine its understanding, enhancing the accuracy of its body modification suggestions.

The output from the deep learning module 204 can serve as the primary output for the classifier 205. The classifier 205 can receive the outputs from the deep learning module 204 and use those outputs to make decisions about what procedures to deliver to the user. Feedback from the classifier 205 can then be used to adjust and refine the outputs from the deep learning module 204. The deep learning module output can act on the rules-based engineering module 206 to inform and update the rule-based engineering module's rule implementation. For example, if the deep learning module 204 identifies that the user has a strong preference for minimally invasive body modifications, then a rule might be triggered to prioritize less invasive procedures in the suggested body modification protocol. Outputs from the deep learning module 204 can be used by the AI-driven search module 202 to inform the AI-driven search module's prioritization of body modification procedures. For example, if the deep learning module 204 determines user interest in a particular procedure, then the AI-driven search module 202 can prioritize identifying and delivering updated resources, research, and expert opinions related to that specific procedure. Speech or text user inputs received from a user (e.g., via the computer sensing module 207) can be transformed into a high-dimensional representation that the natural language processing module 203 can interpret.

The classifier 205 can receive inputs and assign a class label to those inputs. The classifier 205 can take the embedded generated outputs from the deep learning module 204 and make a prediction about the type of procedures a user is likely to be interested in. For example, a 40-year old female may be directed to a cardio workout plan that worked for similar users of the same age and gender. Particularly, if the user is a beginner with limited exercise experience, the classifier 205 might suggest beginner-level cardio routines that are effective yet less strenuous. On the other hand, if the user has an advanced fitness background, the system could recommend more intensive cardio workouts or even cross-training routines tailored for her age group and gender. The classifier 205 can be employed in selecting the general procedures, and the particular procedure from a more general category of procedures, to be delivered to the user after determining the content applicable to the user's unique characteristics.

The classifier 205 can work in tandem with the rules-based engineering module 206. After the classifier 205 makes predictions, but before the predicted procedure is delivered to the user, the predictions may be filtered or adjusted by the rules-based engineering module 206 to ensure the classifier's predictions comply with certain constraints or health restrictions. For example, if a user has previously indicated a back injury, the rules-based engineering module 206 would cross-check the classifier's recommendations to ensure any suggested exercises or procedures don't exacerbate the injury. Additionally, the classifier 205 may interact with the AI-driven search module 202 to focus the AI-driven search module 202 on procedures similar to what the classifier 205 determines is the most relevant procedure to the user. The classifier 205 may use feedback from the natural language processing module 203 to further refine procedure selection. For example, the natural language processing module 203 may interpret the user's input as expressing an interest in particular procedure, then the classifier 205 can prioritize delivery of that particular procedure.

The rules-based engineering module 206, by utilizing predefined logic and constraints (rules), can be employed to influence the machine learning model's output of real-time body modification procedure recommendations. In the context of delivering a body modification procedure, the rules utilized by the rules-based engineering module 206 may relate to what kind of procedures to recommend or not recommend to a user based on a number of predetermined or generated constraints (e.g., based on medical history, age, previous use (effective or ineffective) of a given modality, or safety concerns). For example, the rules may be against recommending certain invasive procedures to individuals with specific medical conditions, or rules about ensuring the system recommends a diverse set of procedures that consider various factors like recovery time and invasiveness. The rules may also apply to edge cases that may not be well-covered by the data used to train the deep learning module 204. The rules-based engineering module 206 may allow for explicitly programmed decisions or behaviors to control the recommended body modification procedures. The rules utilized by the rules-based engineering module 206 may be set in advance, added at a later time, or can be updated periodically to improve procedure recommendations for a user. The rules may apply to ensure the procedure recommendations comply with ethical or medical guidelines established by healthcare professionals, for example.

The rules-based engineering module 206 may use the output from the deep learning module 204 to determine which rules apply to the user. For example, if the deep learning module 204 determines the user has a specific medical condition, then the rules-based engineering module 206 would enforce rules applicable to individuals with that condition. Additionally, the rules-based engineering module 206 may adjust recommendations from the classifier 205. For example, if the classifier 205 recommends a procedure that is deemed too invasive or risky for the user, then the rules-based engineering module 206 could override the classifier's decision. The rules-based engineering module 206 may take data, such as the user's age or medical history, from the computer sensing module 207 and invoke rules applicable to that particular user profile. The rules-based engineering module 206 may interact with the AI-driven search module 202 to help guide the AI-driven search module 202 in finding relevant procedures or alternatives. For example, the machine learning model 200 may employ a rule that the AI-driven search module 202 prioritizes less invasive or popular procedures. The machine learning model 200 may employ rules about certain types of data interpretation (e.g., user preferences, past procedures, feedback) or about interpreting certain user inputs. Thus, the rules-based engineering module 206 may invoke rules that directly operate on the natural language processing module 203.

The AI-driven search module 202 may be used to search either on the internet or other available content (e.g., content stored in a database) to find content most relevant to user's specific interests and needs. The AI-driven search module 202 may use a collaborative filtering technique to identify procedures that similar users have chosen or may use content-based filtering to find procedures that align with the user's past selections or interactions. The AI-driven search module 202 may also use reinforcement learning to continually improve the module's recommendations. For example, the AI-driven search module 202 may, over time, and through interaction with other modules of the machine learning model 200, learn which type of recommended procedures lead to positive user results and prioritize similar recommended procedures in the future. The AI-driven search module 202 may also use real-time user feedback to adjust recommendations instantaneously or substantially instantaneously.

The AI-driven search module 202 may use the outputs from the deep learning module 204 to achieve a comprehensive understanding of the user's body modification preferences. The deep learning module 204 output may help the AI-driven search module 202 rank and retrieve the most relevant body modification procedures to deliver to the user. Additionally, the AI-driven search module 202 may use classification outputs from the classifier 205 to guide the search. For example, a user classified as having a “preference for non-invasive procedures” (e.g., by the classifier 205) might guide the AI-driven search module 202 to prioritize less invasive body modification options. The rules invoked by the rules-based engineering module 206 may modulate the prioritization of procedures retrieved by the AI-driven search module 202. The neural network 201 may provide learned representations that are then used by the AI-driven search module 202 to rank and retrieve the most relevant procedure options. The AI-driven search module 202 may employ the natural language processing module 203 to better understand text-based user inputs.

The natural language processing module 203 may be employed by the machine learning model 200 to understand, interpret, generate, and interact with spoken or written human language. This may include understanding user queries or understanding text-based content. The natural language processing module 203 may be used to understand user feedback or enable text-based user interactions. For example, a user may be able to search for content via a natural language search. Additionally, the natural language processing module 203 may be used to generate human-like text responses that can be used to communicate with the user. This may also include generating the custom body modification protocols delivered by the system. Moreover, the natural language processing module 203 may enable real-time dialogue between the user and the machine learning model 200, allowing the user to ask questions, provide feedback, or change their preferences in a natural, conversational way.

The natural learning processing module 203 may use the deep learning module 204 to process and understand human language inputs. The output from the deep learning module 204 may be used to enhance understanding and generation of natural language. The natural language processing module 203 may use the output from the classifier 205 to tailor the language used in response to a user (e.g., the system may provide more detailed technical responses to a user well-versed user in a particular procedure versus simpler language for a user with a novice level understanding of a suggested procedure). The rules-based engineering module 206 can guide the natural language processing module's 203 use of certain phrases or preferring certain response types. The natural language processing module 203 may use the learned representations from the neural network 201 to better understand the semantics of the user's input and generate appropriate responses. The natural language processing module 203 may help guide the AI-driven search module 202 by interpreting user inquiries and thereby improving the AI-driven search module's 202 search effectiveness. The natural language processing module 203 may gather speech inputs from the computer sensing module 207 and transcribe and interpret those inputs.

In an aspect of the present disclosure, the body modification procedures include an exercise regimen, a diet regimen, a meal plan, a surgical intervention, a non-surgical intervention, a medical intervention, an injection, a facial, a laser treatment, a dental procedure, a salon procedure, a self-care procedure, and/or a cosmetic application procedure.

In an aspect of the present disclosure, the computer 104 is configured to instruct the processor 105 to receive at least one user-preferred body modification procedure. Each of the available body modifications procedures is assigned a first weight. At least one of the user-preferred body modification procedures receives a higher valued second weight. The neural network 201 then determines the available body modification procedures 110 based on the first weight applied to the available body modification procedures and the second weight applied to user-preferred body modification procedure(s).

In an aspect of the present disclosure, the computer 104 is configured to instruct the processor 105 to receive at least one user-disfavored body modification procedure. The user-disfavored body modification procedure(s) are assigned a third weight. The third weight is lower than the first weight. The neural network determines the available modification procedures based on the first weight applied to the body modification procedures and the third weight applied to disfavored body modification procedure(s).

In an aspect of the present disclosure, the computer 104 is configured to instruct the processor 105 to (e.g., by executing computer instructions stored in the member 106) identify at least one body modification procedure that would be unsafe. The computer instructions are configured to instruct the processor 105 to exclude the unsafe body modification procedures from the body modification protocol 111.

In an aspect of the present disclosure, the computer 104 is configured to instruct the processor 105 to (e.g., by executing computer instructions stored in the member 106) receive characteristic data for the user. The characteristic data includes demographic information, medical history, family medical history, allergies or allergy, comorbidities, medication use, pregnancy status, number of children, activity level, and/or body dimensions. The neural network 201 determines the available body modification procedures 110 based on the received user's characteristic data.

In an aspect of the present disclosure, the computer 104 is configured to instruct the processor 105 to (e.g., by executing computer instructions stored in the member 106) receive user ratings for at least one particular portion of the user's body. User rating may be for one or more of the user's: hair, face, skin, facial structure, smile, chin, neck, arm, underarm, chest, back, tummy, hip, flank, thigh, knee, calf, ankle, or feet. As an example, the user rating may be a score ranging from a value of 0-100. The neural network 201 determines the available body modification procedures 110 based on the received user ratings for the portion(s) of the user's body.

Referring particularly to FIG. 1B, the image capture device (e.g., image capture device 121) is configured to capture a number of images of the user's body 103. For example, a 3-dimensional (3-D) model of the user's body may be generated by taking a number of pictures of the user's body from various positions (e.g., Position 1, Position 2, Position 3, and Position 4 in FIG. 1B). As an example, Position 1, Position 2, Position 3, and Position 4 may be at various positions, such as a number of evenly spaced positions) arranged 360 degrees around the user's body 103. Top plan, perspective, aerial, or other views may also be used for capturing images to create the 3-D model of the user's body 107. The computer instructions are configured to instruct the processor 105 to stitch together the images of the user's body to create the 3-dimensional model 107 of the user's body.

In an aspect of the present disclosure, the machine learning module 200 includes a convolutional neural network (CNN) configured to analyze one or more of the images 102 of the user's body 103 to determine the user's current body configuration (see, e.g., 107) and one or more image of the body of another person 108 to determine the desired body configuration for the user.

In an aspect of the present disclosure, the machine learning module 200 includes a chatbot module 112 that may be configured to receive user feedback and provide responses to the user 103. The responses are configured to assist the user 103 in completing the body modification protocol 111 and further tailor the body modification protocol 111 to meet the user's aspirations. Furthermore, the chatbot 112 may be configured to receive user input, such as areas of concern, preferences, and disfavored body modification procedures.

In an exemplary embodiment, the body modification protocol 111 may include a recommendation to incorporate psychological treatment or support into the body modification protocol 111.

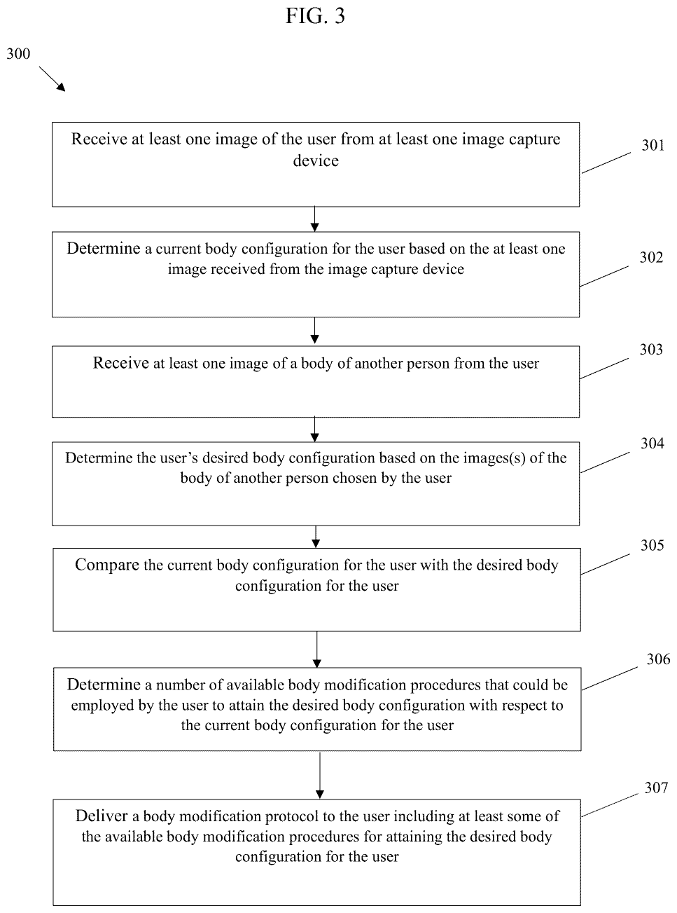

Referring particularly to FIG. 3, a computer-implemented method of generating a body modification protocol 300 includes receiving one or more images of the user's body from at least one image capture device 301. The image or images received from the user are used to determine the user's current body configuration 302. At least one image of a body of another person is received from the user 303. The user's desired body configuration is determined based on the images(s) of the body of another person chosen by the user 304. The user's current body configuration is compared with the desired body configuration for the user by the neural network 305. The available body modification procedures that could be employed by the user to attain the desired body configuration with respect to the current body configuration for the user are determined by the neural network 306. A body modification protocol is delivered to the user 307. The body modification protocol delivered to the user includes at least some of the available body modification procedures for attaining the desired body configuration for the user.

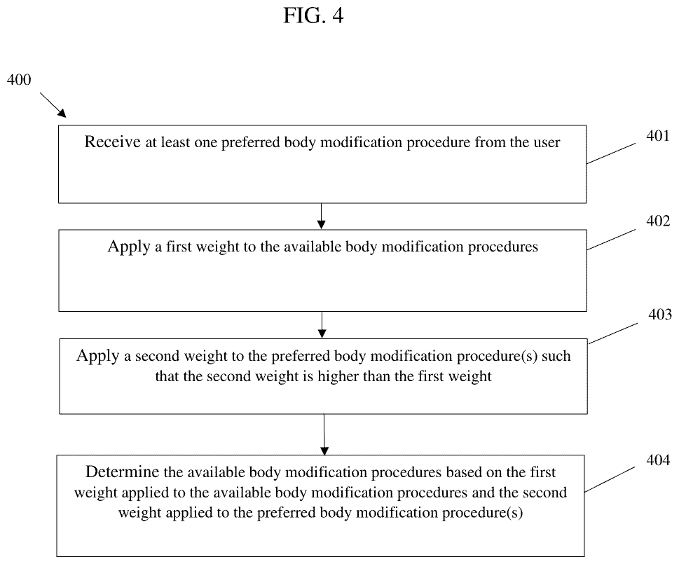

Any of the computer-implemented methods described below with reference to FIGS. 4 to 9 may be applied individually, or in conjunction with (either in whole or in part), the computer implemented methods 300, 1100, or 1200 described herein.

Referring particularly to FIG. 4, a computer implemented method 400 includes receiving at least one preferred body modification procedure from the user 401. The method includes applying a first weight to each of the available body modification procedures 402. The method includes applying a second weight to the preferred body modification procedure(s) 403. The second weight is higher than the first weight. The available body modification procedures are determined, by the neural network, based on the first weight applied to the available body modification procedures and the second weight applied to the preferred body modification procedure(s) 404.

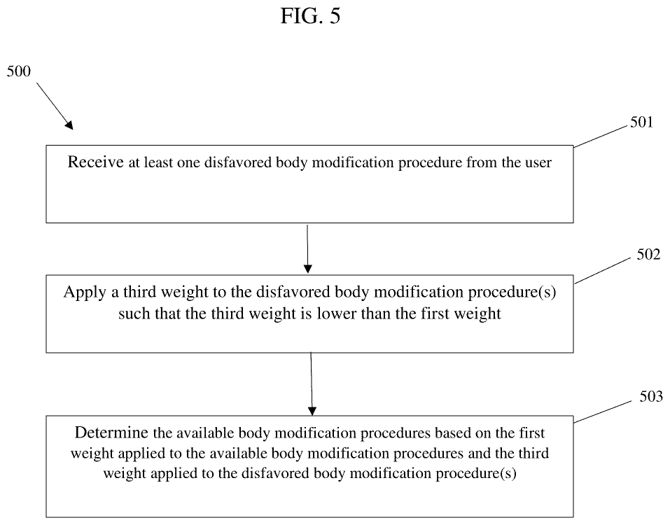

Referring particularly to FIG. 5, a computer implemented method 500 includes receiving at least one disfavored body modification procedure from the user 501. A third weight is applied to the disfavored body modification procedure(s) 502. The third weight is lower than the first weight. The available body modification procedures are determined by the neural network, based on the first weight applied to the available body modification procedures and the third weight applied to the disfavored body modification procedure(s) 503.

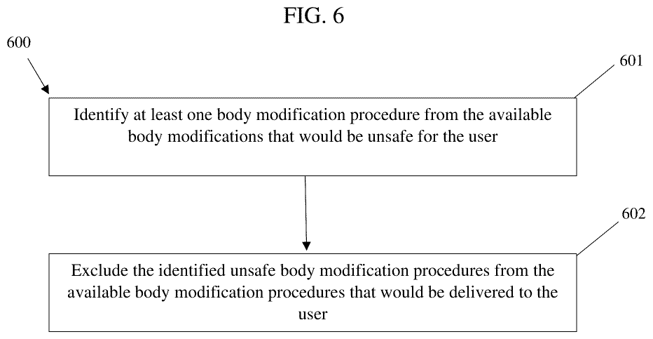

Referring particularly to FIG. 6, a computer implemented method 600 includes identifying at least one available body modification procedure that would be unsafe for the user 601. The method includes excluding the identified body modification procedure that would be unsafe for the user from the body modification protocol delivered to the user 602.

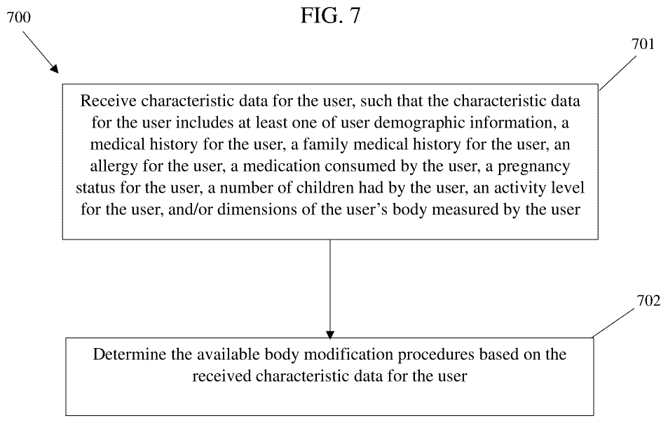

Referring particularly to FIG. 7, a computer implemented method 700 includes receiving characteristic data for the user 701. The received characteristic data for the user includes demographic information, a medical history for the user, a family medical history for the user, an allergy for the user, a comorbidity for the user, a medication consumed by the user, a pregnancy status for the user, a number of children had by the user, an activity level for the user, and/or dimensions of the user's body measured by the user. The available body modification procedures are determined by the neural network based on the received characteristic data for the user 702.

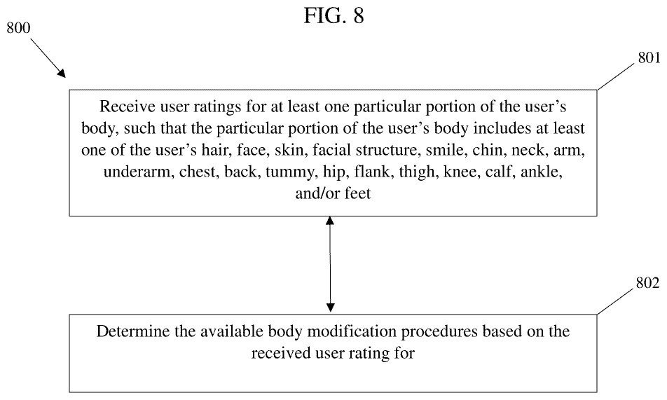

Referring particularly to FIG. 8, a computer implemented method 800 includes receiving user ratings for at least one particular portion of the user's body 801. The particular portion(s) of the user's body includes the user's hair, face, skin, facial structure, smile, chin, neck, arm, underarm, chest, back, tummy, hip, flank, thigh, knee, calf, ankle, and/or feet. The method includes determining the available body modification procedures, by the neural network, based on the received user ratings for the portion(s) of the user's body 802.

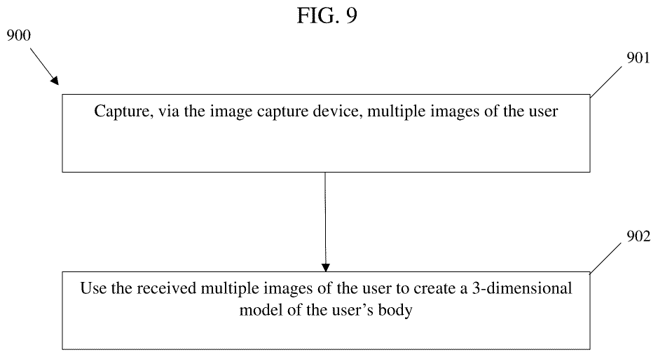

Referring particularly to FIG. 9, a computer implemented method 900 includes capturing, by the image capture device, images of the user's body 901, and using (e.g., stitching together) the images of the user's body to create a 3-dimensional model of the user's body 902.

The image capture device 101 may be a camera array or singular camera in a smartphone, a digital camera, webcam, a video camera, tablet computers, or laptop computers. The image capture device may include multiple zoom features to capture varying degrees of specific user features.

Within the modification protocol system 100, there is the ability to generate multiple output images, each representing a potential outcome post-modification. These images offer the user a visual insight into what the end results of various modifications might look like. To assist the decision-making process, each of these output images is accompanied by a difficulty and/or likelihood of success score. This score provides a quantifiable measure, enabling the user to understand not only the potential look but also the feasibility and complexity associated with achieving each specific outcome. By doing so, users can make more informed decisions, considering both their desired appearance and the practicality or challenges of attaining it.

The body modification protocol system 100 may identify a multitude of distinct body modification protocols. For each identified protocol, the system 100 assigns a probability or likelihood of success, indicating the expected efficacy of the said procedure. Concurrently, a difficulty level is designated, which serves as a measure of the challenges and complexities potentially encountered while pursuing that particular modification. Furthermore, the system calculates and assigns a financial cost and a time cost to each protocol. These cost estimates aid users in gauging the economic and temporal commitments required. Integrating these factors, the system conducts a comprehensive analysis, subsequently generating a set of recommendations. Each recommended option is furnished with a cumulative score, a consolidated metric that considers the likelihood of success, difficulty level, financial implications, and time requirements, thereby offering the user a holistic perspective to make an informed choice.

As an example, delivering the body modification protocol 111 to the user 103 may include providing multiple proposed body modification protocols to the user. Each proposed body modification protocol may include one or more scores ranging from 0-100. The scores may include an indication of a difficulty level for each body modification protocol, a cost associated with each body modification protocol, a level of attainment of a desired body modification if each body modification protocol is completed, and a likelihood that each desired body modification protocol can be achieved. An aggregated or partially aggregated score may also be provided.

For example, a body modification protocol that is difficult to complete, highly expensive, high in level of attainment (i.e., the expected body modification will achieve a look very close to a desired look), and not particularly likely to be achieved may receive scores of 25, 25, 90, and 50, and the corresponding aggregate score would be 47.5.

In another example, a relatively easy, inexpensive, moderate attainment, and high likelihood of success body modification protocol may receive scores of 75, 80, 70, and 90, and the corresponding aggregate score would be 78.75.

Each of the scores or the aggregate scores may be weighted based on user preferences. For example, if a user indicates they are willing to put in any level of effort and financial expense, then cost and difficulty may be weighted lower, and level of attainment may be weighted higher in determining an aggregate score.

Pragmatically, users may not reach a 100% success rate in reaching their desired appearance. Taking this into account and considering empirical observations and user experiences, it's suggested that striving for outcomes nearing perfection, for instance, achieving beyond an 80% success rate, could entail increasingly formidable challenges. Therefore, it may be sensible to suggest protocols that guide users toward realizing approximately 70% to 80% of their desired outcome. This range not only signifies a high degree of accomplishment but also maintains feasibility. The system is calibrated to recognize this gradient of diminishing returns beyond the 80% threshold. Consequently, when generating recommendations, the system prudently takes into account this practical insight, ensuring that the proposed protocols are both effective and realistically attainable for the majority of users.

As an example, the system 100 processes data from a user born Dec. 1, 1990. The user is a 32 year old female, with a height of 5′3″ and weight of 125 lbs, with body measurements detailing her hips, waist, and chest. The system 100 also receives data for several health conditions that the user has reported or that are accessible in a health records database (e.g., a cloud-based database), including psychological conditions, colon cancer, nerve damage, and a prior brain injury. The user provides a self-perception score of 7 out of 10. For the user's facial region, excluding the nose, the user rates her satisfaction at an 8 out of 10 for overall appearance, 7 out of 10 for perceived size and fatness, 9 out of 10 concerning areas of stubborn fat, and a 7 out of 10 regarding hairiness. Skin conditions for this region are rated with elasticity at 8 out of 10, scarring at 6 out of 10, and pigmentation at 6 out of 10. The system 100 further takes into account detailed evaluations for specific facial features like lips, facial hair, and especially the nose, which the user rates highly at 9 out of 10 overall, with specific ratings for size, hairiness, skin elasticity, scarring, pigmentation, wrinkles, freckles, and moles. The user provides assessments for other body areas including the neck, chin, arms, chest, back, hips, and flanks. The neck, chin and arms, for instance, both receive an overall rating of 10 out of 10, with specific ratings for size, stubborn fat, hairiness, and various skin conditions. Notably, for the user's hips and flanks, the user expresses a lower satisfaction at 6 out of 10 overall, with particular concern for cellulite, excess skin, and stretch marks. Other body regions, such as outer and inner thighs, knees, ankles, and teeth, are also inputted into the system with associated ratings. With respect to treatment preferences, the user specifies a timeframe of 1 year, with a budget cap of $15,000. The user indicates a high willingness for surgical intensiveness, rating it at 10 out of 10. Furthermore, the user aspires for a degree of closeness to a new envisioned image at 50%. Included in the delivered body modification protocol (see, e.g., protocol 111 in FIG. 1A) is a post-procedure simulation of the user's appearance after following the body modification protocol and a comparison to her desired appearance. This intricate user data serves as foundational input, allowing the system to generate tailored recommendations that align closely with user's personal preferences and health considerations.

Referring particularly to FIG. 10, a general-purpose computer 1000 is described. The computer (e.g., computer 104 described herein may have the same or substantially the same structure as the computer 1000 or may incorporate at least some of the components of the computer 1000). The general-purpose computer 1000 can be employed to perform the various methods and algorithms described herein. The computer 1000 may include a processor 1001 connected to a computer-readable storage medium or a memory 1002 which may be a volatile type memory, e.g., RAM, or a non-volatile type memory, e.g., flash media, disk media, etc. The processor 1001 may be another type of processor such as, without limitation, a digital signal processor, a microprocessor, an ASIC, a graphics processing unit (GPU), field-programmable gate array (FPGA), or a central processing unit (CPU).

In some aspects of the disclosure, the memory 1002 can be random access memory, read-only memory, magnetic disk memory, solid state memory, optical disc memory, and/or another type of memory. The memory 1002 can communicate with the processor 1001 through communication buses 1003 of a circuit board and/or through communication cables such as serial ATA cables or other types of cables. The memory 1002 includes computer-readable instructions that are executable by the processor 1001 to operate the computer 1000 to execute the algorithms described herein. The computer 1000 may include a network interface 1004 to communicate (e.g., through a wired or wireless connection) with other computers or a server. A storage device 1005 may be used for storing data. The computer 1000 may include one or more FPGAs 1006. The FPGA 1006 may be used for executing various machine learning algorithms. A display 1007 may be employed to display data processed by the computer.

The computer 1000 may employ one or more machine learning models or algorithms or a form of artificial intelligence (see, e.g., machine learning module 200 in FIG. 1A and FIG. 1B) to compare the compare the current body configuration for the user with the desired body configuration for the user, determine a number of available body modification procedures that could be employed by the user to attain the desired body configuration with respect to the current body configuration for the user, generate a personalized body modification protocol, and deliver the personalized body modification protocol to the user.

Generally, the memory 1002 may store computer instructions executable by the processor 1001 to carry out the various functions and algorithms described herein.

The computer 1000 may employ or communicate with various artificial intelligence models, such as one or more machine learning models such as the machine learning model 200 described in more detail above with respect to FIG. 2, in particular.

The neural network 201 may be or may include a convolutional neural network (CNN, or ConvNet), a Bayesian network, a neural tree network, or a support-vector machine (SVM).

While a CNN may be employed, as described herein, other classifiers or machine learning models may similarly be employed. The machine learning model may be trained on tagged data. The trained CNN, trained machine learning model, or other form of decision or classification processes can be used to implement one or more of the methods, functions, processes, algorithms, or operations described herein. A neural network or deep learning model can be characterized in the form of a data structure storing data representing a set of layers containing nodes, and connections between nodes in different layers are formed or created that operate on an input to provide a decision or value as an output.

Machine learning can be employed to enable the analysis of data and assist in making decisions. To benefit from using machine learning, a machine learning algorithm is applied to a set of training data and labels to generate a “model” which represents what the application of the algorithm has “learned” from the training data. Each element (e.g., one or more parameters, variables, characteristics, or “features”) of the set of training data is associated with a label or annotation that defines how the element should be classified by the trained model. A machine learning model predicts a defined outcome based on a set of features of an observation. The machine learning model is built by being trained on a dataset which includes features and known outcomes. There are various types of machine learning algorithms, including linear models, support vector machines (SVM), Bayesian networks, neural tree networks, random forest, and/or XGBoost. A machine learning model may include a set of layers of connected neurons that operate to decide (e.g., a classification) regarding a sample of input data. When trained (e.g., the weights connecting neurons have converged and become stable or within an acceptable amount of variation), the model will operate on new input data to generate the correct label, classification, weight, or score as an output. Other suitable machine learning models may be similarly employed.

As an example, the machine learning model 200 may be trained by accessing data of user behaviors such as product and/or service purchase history, internet browsing history (e.g., from cookies), browsing history within the system described herein, health conditions, and perceived treatment success for prior body modification protocols previously completed in whole or in part by a user. The machine learning model 200 may be trained by accessing data including before and after photos of a user and corresponding ratings from the user related to previously completed body modification protocols.

The machine learning model 200 may be trained by accessing data of user provided ratings related to cost, speed, overall effectiveness, and fidelity to a user's desired protocol outcomes.

The machine learning model 200 may utilize ongoing health and safety information from a peripheral device (see, e.g., peripheral device 211 in FIG. 2), such as a smart watch, a wearable data capturing device, or a body suit configured to capture data from a user's body.

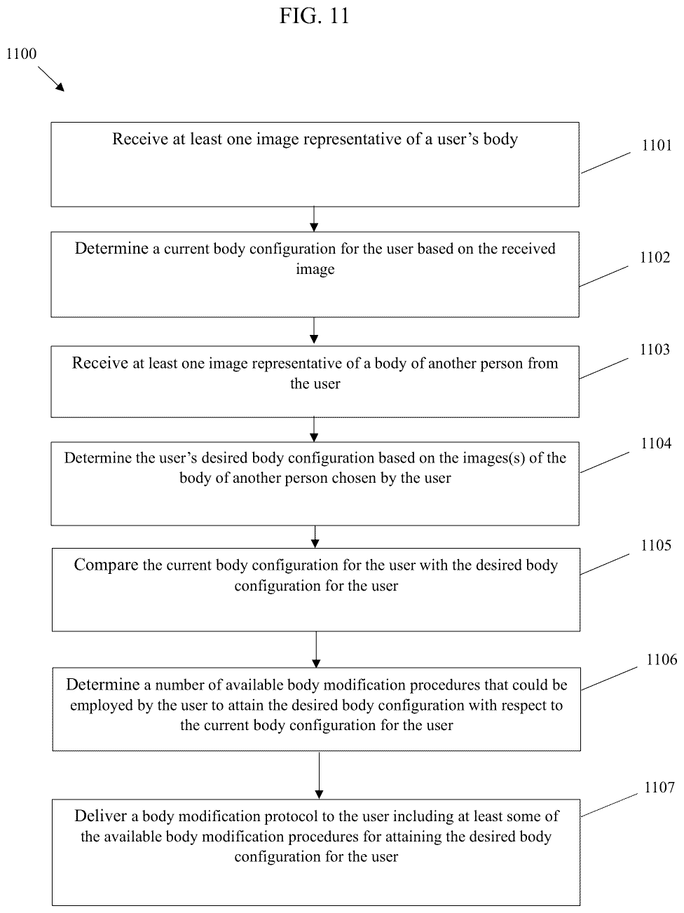

Referring particularly to FIG. 11, a computer-implemented method of generating a body modification protocol 1100 includes receiving one or more images representative of the user's body 1101. As an example, the one or more images representative of the user's body may be received and/or selected (e.g., by the user) from a number of images stored in an image database (see, e.g., image database 212 in FIG. 2). The image or images received from the user are used to determine the user's current body configuration 1102. At least one image of a body of another person is received from the user 1103. The user's desired body configuration is determined based on the images(s) of the body of another person chosen by the user 1104. The user's current body configuration is compared with the desired body configuration for the user by the neural network 1105. The available body modification procedures that could be employed by the user to attain the desired body configuration with respect to the current body configuration for the user are determined by the neural network 1106. A body modification protocol is delivered to the user 1107. The body modification protocol delivered to the user includes at least some of the available body modification procedures for attaining the desired body configuration for the user.

The images stored in the image database 212 may be a number of stock images that are representative of a number of different body types or body configurations. Thus, a user may select one or more representative images of the user's body configuration from a series of stock images, rather than capturing an image of the user's body directly. As an example, a user looking to drop a predetermined amount of belly fat might select an image representative of a body having the corresponding amount of excess belly fat.

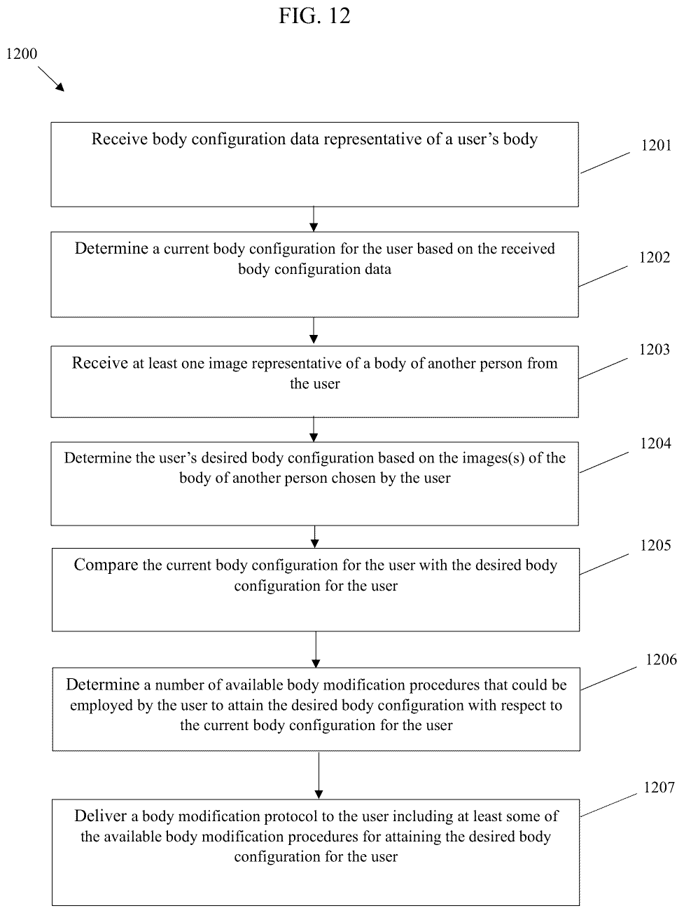

Referring particularly to FIG. 12, a computer-implemented method of generating a body modification protocol 1200 includes receiving body configuration data representative of the user's body 1201. As an example, the body configuration data is generated by the user selecting a series of predetermined options representative of the user's body. The body configuration data received from the user are used to determine the user's current body configuration 1202. At least one image of a body of another person is received from the user 1203. The user's desired body configuration is determined based on the images(s) of the body of another person chosen by the user 1204. The user's current body configuration is compared with the desired body configuration for the user by the neural network 1205. The available body modification procedures that could be employed by the user to attain the desired body configuration with respect to the current body configuration for the user are determined by the neural network 1206. A body modification protocol is delivered to the user 1207. The body modification protocol delivered to the user includes at least some of the available body modification procedures for attaining the desired body configuration for the user.

In exemplary embodiments, the body modification protocols described herein may be generated without the use of any images (e.g., without an image of the user's body and/or without an image of the desired body configuration). For example, a written description of the various body regions or parts can be employed instead or in conjunction with images. As an example, a user may provide a written description or may select a written description of one or more body regions or parts that they are not happy with and/or would like to change. The user may provide ratings of the user's body regions or body parts (e.g., arms are 3 out of 10, legs are 5 out of 10, etc.), and/or the user may provide or select from various body conditions (e.g., thigh is overweight, has unwanted pigmentation; arm is underweight or too thin, acne scarring, bat wings, etc.).

As an example, the body configuration data is generated by the user selecting from a predetermined series of options to describe the user's body in a language-based format (e.g., without the use of images of or representative of the user's body). The body configuration data may be used to generate a model of the user's body or a portion of the user's body, such an 3-D or avatar model of the user's body or the portion of the user's body.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

1. A system for generating a body modification protocol, comprising:
at least one image capture device, wherein the at least one image capture device is configured to capture at least one image of a user's body;
a machine learning model in communication with the at least one image capture device, wherein the machine learning model includes an artificial neural network; and
a computer including a processor and a memory, wherein the computer is in communication with the at least one image capture device and the machine learning model, wherein the memory stores computer instructions configured to instruct the processor to:
receive the at least one image of the user's body from the at least one image capture device;
determine a current body configuration for the user based on the at least one image received from the image capture device,
wherein the current body configuration for the user is determined by generating a 3-dimensional model of the user's body by capturing a plurality of images of the user's body from various positions and stitching together the plurality of images of the user's body;
receive at least one image of a body of another person from the user;
determine a desired body configuration for the user based on the at least one image of the body of another person,
wherein the desired body configuration for the user is determined by generating a 3-dimensional model of the another person's body by capturing a plurality of images of the another person's body from various positions and stitching together the plurality of images of the another person's body;
compare, by the artificial neural network, the current body configuration for the user with the desired body configuration for the user;
determine, by the artificial neural network, a plurality of available body modification procedures that could be employed by the user to attain the desired body configuration with respect to the current body configuration for the user; and
deliver a body modification protocol to the user, wherein the body modification protocol delivered to the user includes at least some of the available body modification procedures of the plurality of available body modification procedures for attaining the desired body configuration for the user,
wherein the computer is in communication with the artificial neural network, wherein the artificial neural network is iteratively trained using training data stored in a training data database, and wherein the iteratively trained artificial neural network generates at least a portion of the body modification protocol.

2. The system of claim 1, wherein the body modification procedures of the plurality of body modification procedures include at least one of an exercise regimen, a diet regimen, a meal plan, a surgical intervention, a non-surgical intervention, a medical intervention, an injection, a facial, a laser treatment, a dental procedure, a salon procedure, a self-care procedure, or a cosmetic application procedure.

3. The system of claim 1, wherein the computer instructions are further configured to instruct the processor to:
receive at least one preferred body modification procedure from the user;
apply a first weight to the body modification procedures of the plurality of available body modification procedures;
apply a second weight to the at least one preferred body modification procedure, wherein the second weight is higher than the first weight; and
determine, by the artificial neural network, the plurality of available body modification procedures based on the first weight applied to the body modification procedures of the plurality of available body modification procedures and the second weight applied to the at least one preferred body modification procedure.

4. The system of claim 3, wherein the computer instructions are further configured to instruct the processor to:
receive at least one disfavored body modification procedure from the user;
apply a third weight to the at least one disfavored body modification procedure, wherein the third weight is lower than the first weight; and
determine, by the artificial neural network, the plurality of available body modification procedures based on the first weight applied to the body modification procedures of the plurality of available body modification procedures and the third weight applied to the at least one disfavored body modification procedure.

5. The system of claim 4, wherein the computer instructions are further configured to instruct the processor to:
identify at least one body modification procedure of the plurality of available body modification procedures that would be unsafe for the user; and
exclude the identified at least one body modification procedure of the plurality of available body modification procedures that would be unsafe for the user from the body modification protocol delivered to the user.

6. The system of claim 1, wherein the computer instructions are further configured to instruct the processor to:
receive characteristic data for the user, wherein the characteristic data for the user includes at least one of user demographic information, a medical history for the user, a family medical history for the user, a comorbidity for the user, an allergy for the user, a medication consumed by the user, a pregnancy status for the user, a number of children had by the user, an activity level for the user, dimensions of the user's body measured by the user; and
determine, by the artificial neural network, the plurality of available body modification procedures based on the received characteristic data for the user.

7. The system of claim 1, wherein the computer instructions are further configured to instruct the processor to:
receive user ratings for at least one particular portion of the user's body, wherein the at least one particular portion of the user's body includes at least one of the user's hair, face, skin, facial structure, smile, chin, neck, arm, underarm, chest, back, tummy, hip, flank, thigh, knee, calf, ankle, or feet; and
determine, by the artificial neural network, the plurality of available body modification procedures based on the received user ratings for the at least one portion of the user's body.

8. (canceled)

9. The system of claim 1, wherein the machine learning model includes a convolutional neural network (CNN) configured to analyze the at least one image of the user's body to determine the current body configuration for the user and the at least one image of the body of another person to determine the desired body configuration for the user.

10. The system of claim 1, wherein the machine learning model includes a chatbot module, and wherein the chatbot module is configured to receive feedback from a user and provide responses to the user, wherein the responses provided to the user are configured to assist the user in completing the body modification protocol.

11. A computer-implemented method of generating a body modification protocol, comprising:
receiving at least one image of the user's body from at least one image capture device;
determining a current body configuration for the user based on the at least one image received from the at least one image capture device,
wherein the current body configuration for the user is determined by generating a 3-dimensional model of the user's body by capturing a plurality of images of the user's body from various positions and stitching together the plurality of images of the user's body;
receiving at least one image of a body of another person from the user;
determining a desired body configuration for the user based on the at least one image of the body of another person,
wherein the desired body configuration for the user is determined by generating a 3-dimensional model of the another person's body by capturing a plurality of images of the another person's body from various positions and stitching together the plurality of images of the another person's body;
comparing, by an artificial neural network of a machine learning model, the current body configuration for the user with the desired body configuration for the user;
determining, by the artificial neural network, a plurality of available body modification procedures that could be employed by the user to attain the desired body configuration with respect to the current body configuration for the user; and
delivering a body modification protocol to the user, wherein the body modification protocol delivered to the user includes at least some of the available body modification procedures of the plurality of available body modification procedures for attaining the desired body configuration for the user
wherein the artificial neural network is iteratively trained using training data stored in a training data database, and wherein the iteratively trained artificial neural network generates at least a portion of the body modification protocol.

12. The computer-implemented method of claim 11, wherein the body modification procedures of the plurality of body modification procedures include at least one of an exercise regimen, a diet regimen, a meal plan, a surgical intervention, a non-surgical intervention, a medical intervention, an injection, a facial, a laser treatment, a dental procedure, a salon procedure, a self-care procedure, or a cosmetic application procedure.

13. The computer-implemented method of claim 11, further including:
receiving at least one preferred body modification procedure from the user;
applying a first weight to the body modification procedures of the plurality of available body modification procedures;
applying a second weight to the at least one preferred body modification procedure, wherein the second weight is higher than the first weight; and
determining, by the artificial neural network, the plurality of available body modification procedures based on the first weight applied to the body modification procedures of the plurality of available body modification procedures and the second weight applied to the at least one preferred body modification procedure.

14. The computer-implemented method of claim 13, further including:
receiving at least one disfavored body modification procedure from the user;
applying a third weight to the at least one disfavored body modification procedure, wherein the third weight is lower than the first weight; and
determining, by the artificial neural network, the plurality of available body modification procedures based on the first weight applied to the body modification procedures of the plurality of available body modification procedures and the third weight applied to the at least one disfavored body modification procedure.

15. The computer-implemented method of claim 14, further including:
identifying at least one body modification procedure of the plurality of available body modification procedures that would be unsafe for the user; and
excluding the identified at least one body modification procedure of the plurality of available body modification procedures that would be unsafe for the user from the body modification protocol delivered to the user.

16. The computer-implemented method of claim 11, further including:
receiving characteristic data for the user, wherein the characteristic data for the user includes at least one of user demographic information, a medical history for the user, a family medical history for the user, a comorbidity for the user, an allergy for the user, a medication consumed by the user, a pregnancy status for the user, a number of children had by the user, an activity level for the user, dimensions of the user's body measured by the user; and
determining, by the artificial neural network, the plurality of available body modification procedures based on the received characteristic data for the user.

17. The computer-implemented method of claim 11, further including:
receiving user ratings for at least one particular portion of the user's body, wherein the at least one particular portion of the user's body includes at least one of the user's hair, face, skin, facial structure, smile, chin, neck, arm, underarm, chest, back, tummy, hip, flank, thigh, knee, calf, ankle, or feet; and
determining, by the artificial neural network, the plurality of available body modification procedures based on the received user ratings for the at least one portion of the user's body.

18. (canceled)

19. The computer-implemented method of claim 11, wherein the machine learning model includes a convolutional neural network (CNN), and wherein the CNN analyzes the at least one image of the user's body to determine the current body configuration for the user and the at least one image of the body of another person to determine the desired body configuration for the user.

20. The computer-implemented method of claim 11, wherein the machine learning model includes a chatbot module, and wherein the chatbot module receives feedback from a user and provides responses to the user, wherein the responses provided to the user are configured to assist the user in completing the body modification protocol.
